(19) United States
(12) Patent Application Publication
(10) Pub. No.: US 2013/0055470 A1
(43) Pub. Date: Feb. 28, 2013

(54) GENES ENCODING PROTEINS ABLE TO ENHANCE HEAT RESISTANCE OF PLANTS AND MICROORGANISMS AND USES THEREOF

(71) Applicant: Yi Yang, Chengdu (CN)

(72) Inventor: Yi Yang, Chengdu (CN)

(73) Assignee: SICHUAN BIODESIGN GENE ENGINEERING CO.,LTD, Chengdu, Sichuan (CN)

(21) Appl. No.: 13/575,738

(22) PCT Filed: Jan. 28, 2011
(86) PCT No.: PCT/CN2011/070745

§ 371 Date: Nov. 2, 2012

(30) Foreign Application Priority Data
Jan. 28, 2010 (CN) 201010300890.X

(51) Int. Cl. C12N 15/52; C12N 9/00; C12N 9/96; C12N 15/82; C12N 15/63; C12N 5/10; C12N 1/21; C12N 15/70; C12N 15/62; C12N 15/29

(52) U.S. Cl. 800/289; 536/23.2; 435/183; 435/188; 536/23.6; 435/320.1; 435/419; 435/252.33; 435/471

(57) ABSTRACT

Genes encoding proteins able to enhance heat resistance of plants and microorganisms, proteins encoded by the same and uses thereof are provided, wherein the genes contain nucleotide sequences encoding the following peptide fragments: N′-CRICQE X₇₋₄₅ PCAC X₆ AHR X₁ CVQ X₁₃₋₂₇-C′, wherein X represents any amino acid and the subscript represents the number of amino acid.

### TECHNICAL FIELD

The present invention belongs to molecular biology field. And more specifically, the invention relates to genes encoding proteins able to enhance heat resistance of plants and microorganisms, proteins encoded by the same and uses thereof.

### BACKGROUND ART

Because of the increased CO₂ emission, greenhouse effect on the earth is growing worse and leads to global warming. It is estimated that the global average temperature will increase by 1.4-5.8° C. in the next 100 years. Global warming gradually deteriorates the agricultural ecological environment. It is predicted that climate warming may lead to 17% of crop yield reduction. One research from IRR1 (International Rice Research Institute) proved that during 1998-2003, the crop yield was decreased by 10% with the temperature elevated by 1° C. In China, experts believe that by 2050, the nationwide average temperature will increase by 2.2° C. Plants growing under natural conditions are all affected by the elevated temperature and grow more slowly. Some major crops, such as rice and corn, are especially easy to be influenced by hot weather during earing and postulation, and result in crop yield reduction. On the other hand, according to FAO (Food and Agriculture Organization of the United Nations), the world population will exceed 10 billions by 2050. With further increased world population, there will be more and more pressure on agriculture and worldwide food shortage will be a long-lasting problem. Being affected by global warming, lots of herbaceous plants will grow more slowly and even die, thus breaking ecosystem balance. Therefore, scientists all over the world are taking great efforts in searching for heat-tolerance relevant plants genes. So far from now, only a few heatshock protein genes and transcription factors thereof are found to be relevant with heat tolerance, while none of a single gene was reported to be capable of increasing heat tolerance of bacteria and plants. It is now of great demands in the art to find genes able to enhance heat resistance of plants.

Ubiquitin, which is named because of its wide distribution in various kinds of cells, is a highly conserved polypeptide consisting of 76 amino acid residues. Ubiquitin-labeled proteins will be specifically recognized and rapidly degraded. As is shown in studies, ubiquitin-mediated protein degradation is an important physiological and biochemical process for organisms to maintain normal growth as well as growth under stresses.

Ubiquitination of protein mainly occurs to the side chain of lysine residue and is usually a multimerization process. Multi-ubiquitinated proteins will be recognized and degraded by proteasome. There are three crucial enzymes that mediate such a multi-ubiquitination process, including ubiquitin activating enzyme E1, ubiquitin conjugating enzyme E2 and ubiquitin ligase E3. The mechanism of those enzymes is now substantially illustrated as follows: firstly, a thioester bond is formed between the Cys residue of the active center of E1 and the C-terminal of ubiquitin, to activate the individual free ubiquitin (wherein ATP is needed); then, the activated ubiquitin is delivered from E1 to E2; finally, specific substrate and E2 are collected by E3, which mediates the transferring of ubiquitin from E2 to the target protein. Since the process of recognition and degradation of ubiquitinated protein by proteasome is non-specific, the role of E3 is extremely important in the protein degradation process to determine the specificity of the reaction. Usually, ubiquitination involves one E1, several E2 and many E3. The E3 family discovered so far has two types, including the majority of RING-finger family and the HECT family.

### DISCLOSURE OF THE INVENTION

The technical problem to be solved in the invention is to provide genes (also called heat-resisting genes in the invention) encoding proteins (also called heat-resisting proteins in the invention) that are able to enhance heat resistance of plants and microorganisms.

Said genes encoding proteins that are able to enhance heat resistance of plants or microorganisms comprise nucleotide sequences encoding peptides with structures as shown in

N′-CRICQE X₇₋₄₅ PCAC X₆ AHR X₁ CVQ X₁₃₋₂₇-C′ (SEQ ID NO: 68), wherein X represents any amino acid and the subscript represents the number of amino acid.

Furthermore, said peptides have amino acid sequences of:

N′-CRICQEED X₃₋₂₀ NL X₃₋₂₀ PCAC X₂ SLK X₁ AHR X₁ CVQRWC X₁₀₋₂₄ (SEQ ID NO: 69), wherein X represents any amino acid residue and the subscript represents the number of amino acid.

Wherein, the forementioned proteins are ubiquitin ligases (E3).

Wherein, the forementioned proteins are transmembrane proteins, and comprise transmembrane domains.

Wherein, the forementioned genes are derived from plant genomes.

Wherein, the forementioned plants are selected from arabidopsis (Arabidopsis thaliana L.), rice (Oryza sativa L.), corn (Zea mays L.) or castor oil plant (Ricinus communis L.).

Wherein, the forementioned genes (1): comprise nucleotide sequences shown in SEQ ID NO: 39, SEQ ID NO: 1, SEQ ID NO: 45, SEQ ID NO: 47 or SEQ ID NO: 57;

Or (2): the forementioned genes comprise nucleotide sequences derived from substitution, deletion or addition of at least one nucleotide in any one of said nucleotide sequences in (1), while encoding proteins with the same or similar function.

Furthermore, said genes comprise (1): nucleotide sequences of sites 158-1018 in SEQ ID NO: 57, sites 96-848 in SEQ ID NO: 45, sites 291-1127 in SEQ ID NO: 47, or sites 68-946 in SEQ ID NO: 39;

Or (2): said genes comprise nucleotide sequences derived from substitution, deletion or addition of at least one nucleotide in any one of said nucleotide sequences in (1), while encoding proteins with the same or similar function.

The invention also provides proteins encoded by the forementioned genes.

The invention further provides proteins that enhance the heat resistance of plants or microorganisms. Such proteins comprise peptides with structures as shown in:

N′-CRICQE X₇₋₄₅ PCAC X₆ AHR X₁ CVQ X₁₃₋₂₇-C′, wherein X represents any amino acid and the subscript represents the number of amino acid.

Furthermore, said peptides have amino acid sequences of:

N′-CRICQEED X₃₋₂₀ NL X₃₋₂₀ PCAC X₂ SLK X₁ AHR X₁ CVQRWC X₁₀₋₂₄-C′, wherein X represents any amino acid.

Wherein, the forementioned proteins are transmembrane proteins comprising transmembrane domains. The forementioned proteins comprise 1-6 transmembrane domains. Furthermore, said protein comprises 2-3 transmembrane domains.

Wherein, the structure of the forementioned transmembrane domain is at least one of N′-A X₂₋₆ CRS X₂₋₈ LIL X₂₋₄ LL X₁₋₄ LR X₁₋₁₀-C′ (SEQ ID NO: 70), or N′-L X₂₋₄ R X₁₋₅ GFLL X₁₋₇ YIMAW X₁₋₁₅-C′ (SEQ ID NO: 71), wherein X represents any amino acid and the subscript represents the number of amino acid. Wherein, there is a flexible connection between each of the forementioned transmembrane domains. Wherein, the forementioned transmembrane domain is close to the C-terminal. Wherein, the forementioned transmembrane domain is flexibly connected to a zinc finger domain. Wherein, the forementioned protein also has a signal peptide at the N-terminal. Wherein, the forementioned signal peptide is a membrane localization signal peptide.

Wherein, the forementioned proteins are ubiquitin ligases.

Wherein, the forementioned proteins are derived from plants.

Wherein, the forementioned proteins comprise amino acid sequences encoded by nucleotide sequences shown in SEQ ID NO: 1 (the amino acid sequence encoded by which is shown in SEQ ID NO: 2), sites 158-1018 in SEQ ID NO: 57 (the amino acid sequence encoded by which is shown in SEQ ID NO: 58), sites 96-848 in SEQ ID NO: 45 (the amino acid sequence encoded by which is shown in SEQ ID NO: 46), sites 291-1127 in SEQ ID NO: 47 (the amino acid sequence encoded by which is shown in SEQ ID NO: 48) or sites 68-946 in SEQ ID NO: 39 (the amino acid sequence encoded by which is shown in SEQ ID NO: 40);

Or (2): said protein comprises amino acid sequences derived from substitution, deletion or addition of at least one amino acid in any one of amino acid sequences in (1), while possessing with the same or similar function.

The invention also provides genes encoding the forementioned heat-resisting proteins.

The invention also provides peptides comprising domains as shown in N′-CRICQE X₇₋₄₅ PCAC X₆ AHR X₁ CVQ X₁₃₋₂₇-C′, wherein X represents any amino acid and the subscript represents the number of amino acid.

Furthermore, said domains comprise amino acid sequences of N′-CRICQEED X₃₋₂₀ NL X₃₋₂₀ PCAC X₂ SLK X₁ AHR X₁ CVQRWC X₁₀₋₂₄-C′, wherein X represents any amino acid residue and the subscript represents the number of amino acid.

Wherein, the forementioned peptides are derived from plants. Said plants may be selected from arabidopsis, rice, corn or castor oil plant.

Wherein, the forementioned peptides may form zinc fingers, i.e., domains as shown in N′-CRICQE X₇₋₄₅ PCAC X6 AHR X1 CVQ X₁₃₋₂₇-C′ or N′-CRICQEED X₃₋₂₀ NL X₃₋₂₀ PCAC X₂ SLK X₁ AHR X₁ CVQRWC X₁₀₋₂₄-C′ may form zinc fingers, wherein X represents any amino acid and the subscript represents the number of amino acid.

Wherein, the forementioned peptides may enable proteins to enhance the heat resistance of plants or microorganisms.

Furthermore, the forementioned stress resistance is heat resistance. Moreover, said protein is an ubiquitin ligase.

Moreover, the forementioned peptides comprise sequences as follows:

(1): amino acid sequences shown in SEQ ID NO: 63, SEQ ID NO: 64, SEQ ID NO: 65, SEQ ID NO: 66 or SEQ ID NO: 67;

Or, amino acid sequences derived from substitution, deletion or addition of at least one amino acid in any one of amino acid sequences in (1), while possessing with the same or similar function.

Wherein, the forementioned “at least one” means one or several (less than 10). The forementioned “same or similar function” means the ability to enhance the heat resistance of plants or microorganisms.

The invention also provides nucleotide sequences encoding the forementioned peptides.

The invention also provides a method of modifying genes encoding non-heat-resisting proteins into those encoding heat-resisting ones, comprising the step of inserting forementioned peptide-encoding nucleotide sequences into genes encoding non-heat-resisting proteins, or substituting one section of nucleotide sequences therein. Certainly, such an insertion or substitution occurs between, not inside, codons, and therefore the modified gene still has the ability to encode an entire protein. That is to say, the method comprise the step of inserting forementioned peptide-encoding nucleotide sequences into genes encoding non-heat-resisting proteins, or substituting one section of nucleotide sequences therein; and such an insertion or substitution occurs between codons, therefore the modified gene still has the ability to encode an entire protein.

Wherein, the forementioned substituted nucleotide sequence is the one that encoding zinc finger domain. That is to say, the nucleotide sequence substituted from the forementioned gene encoding non-heat-resisting protein is the one that encoding zinc finger domain.

Wherein, the forementioned non-heat-resisting proteins are ubiquitin ligases (E3 ligase).

Based on the forementioned parts, the invention further provides a method for enhancing the heat resistance of plants, or a method for preparing plants with high heat resistance. Such a method includes steps as follows:

a). operably linking the forementioned gene encoding heat-resisting protein into the downstream of the expression and regulation sequence of a vector, to form a recombinant vector for the expression of said gene;

b). transforming the recombinant vector of step a) into plant cells; and

c). selecting and obtaining transformed cells, followed by regenerating the transformed cells to form transgenic plants or offsprings thereof, said offsprings include plant seeds or plant tissues. Furthermore, the stress resistance in the forementioned method is heat resistance.

The invention also provides vectors comprising the forementioned gene sequences or nucleotide sequences, wherein, said vectors are expression vectors.

Furthermore, said expression vectors are eukaryotic expression vectors.

The invention also provides host cells comprising the forementioned vectors. Said host cells are mainly plant cells or microorganisms.

Said recombinant vectors of the invention are obtained by inserting genes into vectors. The forementioned vectors may be selected from various vectors known in the art, especially eukaryotic expression vectors (e.g. pBI121 or pCAMBIA2301). Host plant cells can be transformed with the forementioned recombinant vectors of the invention, and the transformed cells can be screened under various forementioned stresses.

In the present invention, statements like “nucleotide sequences derived from substitution, deletion or addition of at least one nucleotide in SEQ ID NO: 1” generally refer to nucleotide sequences encoding polypeptides that possess with the activity of the protein encoded by SEQ ID NO: 1, and the degenerate sequences thereof. Said degenerate sequences are sequences with one or more codons being substituted by degenerate codons encoding the same amino acid. Because of the codon degeneracy, a degenerate sequence exhibiting as low as 89% of homology to SEQ ID NO: 1 may encode the sequence encoded by SEQ ID NO: 1 as well. The term of “nucleotide sequences derived from substitution, deletion or addition of at least one nucleotide in SEQ ID NO: 1” also includes nucleotide sequences that can hybridize with SEQ ID NO: 1 under moderate stringent conditions, preferably under high stringent conditions. The term also includes nucleotide sequences exhibiting at least 80%, more preferably at least 90%, and most preferably at least 95% of homology to the nucleotide sequence of SEQ ID NO: 1. The same function in the present invention means enhancing heat resistance of plants or microorganisms.

The term also includes variants of the open reading frame sequence of SEQ ID NO: 1 that can encode proteins having the same function as natural SEQ ID NO: 1. Such variants include (but not limit to): deletion, insertion and/or substitution of several nucleotides (normally 1-90, preferably 1-60, more preferably 1-20 and most preferably 1-10), as well as addition at 5′ and/or 3′ terminals of several nucleotides (normally less than 60, preferably less than 30, more preferably less than 10 and most preferably less than 5).

In the present invention, statements like “amino acid sequences derived from substitution, deletion or addition of at least one amino acid in said amino acid sequences” include, but not limit to, deletion, insertion and/or substitution of several amino acids (normally 1-50, preferably 1-30, more preferably 1-20 and most preferably 1-10), as well as addition at C- and/or N-terminals of several amino acids (normally less than 20, preferably less than 10 and more preferably less than 5). For example, in said proteins, substitutions by amino acids with similar properties usually do not change the function of the protein. Another example is that the addition at C- and/or N-terminals of one or more amino acids usually does not change the protein function, either. The term also includes active fragments and derivatives of said proteins. The same function in the present invention means enhancing heat resistance of plants or making other proteins capable of enhancing heat resistance of plants or microorganisms.

Statements like “amino acid sequences derived from substitution, deletion or addition of at least one amino acid in said amino acid sequences” also include, but not limited to polypeptides having at most 10, preferably at most 8 and more preferably at most 5 of amino acids substituted by those with similar properties, namely, polypeptides with conserved variation. Such polypeptides with conserved variation are obtained most preferably from substitutions according to Table 1.

TABLE 1
Substitutions of amino acids

| Original | Representative | Preferable |
|---|---|---|
| residues | substitutions | substitutions |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also includes analogues of the claimed proteins or polypeptides. Such analogues may be different from the natural polypeptide of SEQ ID NO: 2 in amino acid sequence, or in modifications that will not change the sequence, or both. Such polypeptides include natural or induced genetic variants. The induced variants could be obtained with various techniques, such as random mutagenesis by radiation or exposing to mutagens, as well as site-directed mutagenesis or other known molecular biology techniques. Said analogues also include those comprising residues different from natural L-amino acids (e.g. D-amino acids), and those comprising non-naturally existed or synthetic amino acids (e.g. β, γ-amino acids). It should be appreciated that proteins or polypeptides of the present invention are not limited to the above-mentioned exemplary representatives.

The modification (normally without changing the primary structure) includes chemical derivatization of polypeptides in vivo or in vitro, such as acetylation or carboxylation. The modification also includes glycosylation, such as polypeptides produced from glycosylation in polypeptide synthesis, or from processing or reprocessing steps, which could be achieved by exposing polypeptide to enzymes performing glycosylation (e.g. glycosylase or deglycosylase of mammals). The modification also includes sequences comprising phospho-amino acid residues (e.g. phosphotyrosine, phosphoserine or phosphothreonine). Also included are polypeptides modified to have the property of enhanced proteolysis resistance or improved solubility.

Said “operably-linked” in the present invention refers to conditions that certain parts of linear DNA sequence can affect activities of the others on the same linear DNA sequence. For example, if a signal peptide DNA is expressed as a precursor and participates the secretion of a polypeptide, then the signal peptide DNA (that secretes leading sequence) is operably-linked to the polypeptide DNA; if a promoter controls the transcription of a sequence, then it is operably-linked to the coding sequence; or if a ribosome binding site is placed at a position that it could be translated, then it is operably-linked to a coding sequence. Generally speaking, “operably-linked” means being contigeous, and for the secretion of leading sequence it means being contigeous in reading frames.

The invention shows advantages as follows: it provides genes encoding heat-resisting proteins, and proteins encoded thereof; it also provides polypeptides functioning similarly, and genes thereof; and it further provides uses of these genes and proteins for the enhancement of heat resistance of plants and microorganisms. The invention provides new alternative genes for the enhancement of heat resistance of plants and microorganisms and therefore promises a good application prospect.

### DESCRIPTION OF DRAWINGS

FIG. 1 Growth curve of E. coli transformed with AtTR1 gene and mutants thereof. In the figure, AtTR1 represents E. coli transformed with over expressed AtTR1 gene, MT1-6 respectively represents E. coli transformed with over expressed mutant AtTR1 genes, and WT is non-transgenic E. coli.

FIG. 2 Growth curve of E. coli transformed with RcTR1 gene, the homologous gene of AtTR1 in castor oil plant.

FIG. 3 Growth curve of E. coli transformed with OsTR1-1 and OsTR1-2 genes, the homologous genes of AtTR1 in rice.

FIG. 4 Growth curve of E. coli transformed with ZmTR1 gene, the homologous gene of AtTR1 in corn.

FIG. 5 Growth curve of E. coli with the transmembrane domain of AtTR1 protein substituted or deleted.

FIG. 6 Growth curve of E. coli with the transmembrane domain of OsTR1-1 protein substituted or deleted.

FIG. 7 Growth curve of E. coli with the transmembrane domain of ZmTR1 protein substituted or deleted.

FIG. 8 Detection of E3 ubiquitin ligase activity of AtTR1 protein (+ for added; − for not added)

FIG. 9 Detection of E3 ubiquitin ligase activity of OsTR1-1 protein (+ for added; − for not added)

FIG. 10 Detection of E3 ubiquitin ligase activity of ZmTR1 protein (+ for added; − for not added)

### EMBODIMENTS

The following experiments were performed according to, if not specially defined, routine conditions known by those skilled in the art, such as in Sambrook and Russell, Molecular Cloning: A Laboratory Manual (3^{rd} ed). (New York: Cold Spring Harbor Laboratory Press, 1989), or conditions suggested by manufacturers. In the following examples, vectors pET28, pGEX-2T, pGEM-T and strain BL21 were purchased from Qiagen corp., and strain EHA105 and vector pBI121 were from Clontech corp.

In recent years, it is a hotspot to improve plants with genetic engineering techniques, and there is one choice to enhance stress resistances of plants and cultivate plant lines of resistance by genetic engineering techniques. However, there is seldom report of a single gene that could comprehensively enhance several of resistances for plants and microbes to abiotic stresses. The inventor has cloned an AtTR1 gene from arabidopsis that could enhance heat resistance of plants.

Study showed that, protein expressed by said AtTR1 gene is possessed with ubiquitin E3 ligase activity of the ubiquitin protein system; and by transforming arabidopsis, brassica, and E. coli with the AtTR1 gene, heat resistances in all transgenic organisms were enhanced. Therefore, the gene might be used in the enhancement of heat resistance of plants and microorganisms to reduce the crop failure from high temperature, as well as lowering costs, thus promising economic significance and application prospect.

Mutations were performed to some conserved amino acids of AtTR1 protein in order to study the mechanism of the AtTR1 gene and search for critical functional sites of AtTR1 protein.

TABLE 2
Alignment of AtTR1 and the MARCH family proteins

| Proteins | Critical sequences (the Ring structures) |
|---|---|
| AtTR1 | 69 CRICQE.EDSTKNLEAPCACNGSLKYAHRKCVQRWCNEKGDITCEIC 114 |
| MARCH2 | 64 CRICHE.GANGECLLSPCGCTGTLGAVHKSCLEKWLSSSNTSYCELC 109 |
| MARCH4 | 163 CRICFQ.GPEQGELLSPCRCDGSVKCTHQPCLIKWISERGCWSCELC 208 |
| MARCH6 | 9 CRVCRSEGTPEKPLYHPCVCTGSIKFIHQECLVQWLKHSRKEYCELC 55 |

The alignment (see Table 2) of protein structures showed that AtTR1 protein shared similar structure with MARCH (Membrane Associated Ring-CH), a protein involved in the human immune regulation, wherein both proteins comprised a typical Ring-CH domain. Mutations were performed at several critical sites shown in the alignment. Results showed that these mutants exhibited changed heat resistance, higher or lower than that of the non-transgenic, but all lower than that of trans-AtTR1 gene, showing that these critical sites determined the activity of AtTR1 protein of enhancing stress resistance of organisms.

Based on the forementioned study results, several homologous genes of the AtTR1 gene were found in other plants. Said genes comprised some highly conserved regions and they too were possessed with the function of enhancing heat resistance of plants (see Table 3 for core sequence analysis).

TABLE 3
Alignment of functional sites of AtTR1 and homologous proteins thereof

| | |
|---|---|
| Arabidopsis | 60 EEPLLQSVECRICQEEDSTKNLEAPCACNGSLKYAHRKCVQRWCNEKGDITCEICHQPYQ 119 |
| Castor oil | 60 EEPLLQTMECRICQEEDSINNLEAPCACSGSLKFAHRKCVQRWCNEKGDITCEICHQPYQ 119 |
| plant | |
| Corn | 59 E.PLIQAAECRICQEEDSVKNLEKPCACSGSLKYAHRACVQRWCNEKGDTTCEICHEEYK 118 |
| Rice 1 | 23 TGALIGMVECRICQEEDLAKNLESPCACSGSLKYAHRECVQRWCNEKGDIICEICHVSYK 82 |
| Rice 2 | 50 EEPLIQAAECRICQEEDSlKNLEKPCACSGSLKYAHRACVQRWCNEKGDITCEICHEQYK 109 |
| | RINGv region |
| Arabidopsis | 180 AAFCRSAALILMALLLLRDALNLTTNPDDEDDPTAFFSLFLLRAAGFLLPCYIMAWAIGI 239 |
| Castor oil | 181 AAFCRSAALILMALLLLRHAMSLTG..DSDEDASTFFSLFLLRAAGFLLPCYIMAWAISI 238 |
| plant | |
| Corn | 175 AAFCRSAALILMALLLLRHALSMSDNEGNDDDASTIFSLFLLRAAGFLLPCYIMAWIFSI 234 |
| Rice 1 | 145 AAFCRSIFLILMALLLLRHTLTTTSSDDE.DDASAIFSLFLLRAAGFLLPCYIMAWAISI 203 |
| Rice 2 | 163 AAFCRSAALILMALLLLRHALSISDNEG.DDDASTMFSLFLLRAAGFLLPCYIMAWIFSI 221 |
| | TMD transmembrane domain TMD transmembrane domain |

It was confirmed that a conserved RINGv region comprising amino acid sequence as follows was essential for heat resistance:

N-terminal-C RIC QE X₇₋₄₅ PC AC X₆ AHR X₁ CVQ X₁₃₋₂₇-C-terminal

Furthermore, said region comprised:

N′-C MC QEED X₃₋₂₀ NL X₃₋₂₀ PC AC X₂ SLK X₁ AHR X₁ CVQRWC X₁₀₋₂₄-C′

The italic amino acids referred to sites that could not be changed according to the mutation experiment, and if mutated, the heat resistance would be lost or reduced; wherein X represents any amino acid (preferably natural amino acids) and the subscript represents the number of amino acid.

TABLE 4
Patterns of common zinc finger proteins

| 1 | | 2 | | 3 | | 4 | | 5 | | 6 | | 7 | | 8 | |
|---|---|---|---|---|---|---|---|---|---|---|---|---|---|---|---|
| C | X₂ | C | X₉₋₃₉ | C | X₁₋₂ | H | X₂₋₃ | C | X₂ | C | X₄₋₄₈ | C | X₂ | C | RING |
| C | X₂ | C | X₉₋₃₉ | C | X₁₋₂ | H | X₂₋₃ | H | X₂ | C | X₄₋₄₈ | C | X₂ | C | RING-H2 |
| C | X₂ | C | X₁₀₋₄₅ | C | X₁ | C | X₇ | H | X₂ | C | X₁₁₋₂₅ | C | X₂ | C | RINGv(RING-CH) |
| C | X₁₋₂ | C | X₇₋₂₁ | C | X₂₋₄ | C | X₄₋₅ | H | X₂ | C | X₁₀₋₄₆ | C | X₂ | C | PHD-LAP |

It was shown in Table 4 that, the amino acids at forementioned 1, 2 and 5, 6 sites formed a zinc finger, wherein x could be an arbitrary amino acid. Line 3 showed a specific zinc finger (RINGv) of proteins of the invention, which was specific in plants, and closely related to heat resistance of proteins of the invention.

### Example 1

### Cloning of Heat-Resisting AtTR1 Gene from Arabidopsis and Construction of Over Expression Recombinant Vector

1. The AtTR1 gene was selected from arabidopsis with a nucleotide sequence as shown in SEQ ID NO: 1 from the sequence list. Primers were designed according to the nucleotide sequence as shown in SEQ ID NO: 1, as follows:

Upstream primer (SEQ ID NO: 3):
5′-ATGGCTGATCATTTGAGTTTATGT-3′,
Downstream primer (SEQ ID NO: 4):
5′-TCAAACTGGTGTTGGGACATTGGATA-3′.

The nucleotide sequence was then amplified by PCR from arabidopsis cDNA. The PCR product was purified (see manuals of PCR product purification of Qiagen corp.) and sequenced to obtain gene fragments of the sequence of SEQ ID NO: 1.

2. Primers were designed according to the nucleotide sequence of SEQ ID NO: 1, as follows:

Upstream primer (SEQ ID NO: 5):
5′-CGCGGATCC ATGGCTGATCATTTGAGTTTATGT-3′,
Downstream primer (SEQ ID NO: 6):
5′-CCGGAGCTC TCAAACTGGTGTTGGGACATTGGATA-3′.

Restriction enzyme cutting sites were added by PCR to the entire SEQ ID NO: 1 nucleotide sequence amplified from arabidopsis cDNA. The PCR product was purified (see manuals from Qiagen corp.), enzymatic digested by BamH1 and Sac1, recovered from the gel, and ligated into the vector pBI121 (ligation sites: BamH1 and Sac1), to obtain the over expressed recombinant plasmid of SEQ ID NO: 1.

### Example 2

### Site-Directed Mutagenesis of AtTR1 Gene and the Construction of Over Expression Recombinant Vectors Comprising Mutated Genes

1. Several sites were randomly selected and primers comprising mutated sites were designed according to SEQ ID NO: 1 (mutations were indicated by underlines), to construct sequences of site-directed mutagenesis (Stratagene Corp., QuikChange® Site-Directed Mutagenesis Kit).

Primers for amino acid sequence comprising mutation at site 69:

Upstream primer (SEQ ID NO: 7):
5′-TTCTC CAATC TGTTG AGAGT CGTAT TTGCC-3′
Downstream primer (SEQ ID NO: 8):
5′-GGCAA ATACG ACTCT CAACA GATTG GAGAA-3′

Primers for amino acid sequence comprising mutation at site 72:

Upstream primer (SEQ ID NO: 9):
5′-AGTGT CGTAT TGGCC AAGAG GAAGA TAGTA CT-3′
Downstream primer (SEQ ID NO: 10):
5′-AG TACTA TCTTC CTCTT GGCCA ATACG ACACT-3′

Primers for amino acid sequence comprising mutation at site 85:

Upstream primer (SEQ ID NO: 11):
5′-CTTGA AGCTC CTAGT GCTTG TAA-3′
Downstream primer (SEQ ID NO: 12):
5′-TTA CAAGC ACTAG GAGCT TCAAG-3′

Primers for amino acid sequence comprising mutation at site 87:

Upstream primer (SEQ ID NO: 13):
5′-GAAGC TCCTT GTGCT CGTAA TGGTA GTTTA AA-3′
Downstream primer (SEQ ID NO: 14):
5′-TT TAAAC TACCA TTACG AGCAC AAGGA GCTTC-3′

Primers for amino acid sequence comprising mutation at site 95:

Upstream primer (SEQ ID NO: 15):
5′-TAAAG TATGC TAACC GCAAG TGTGT TC-3′
Downstream primer (SEQ ID NO: 16):
5′-GA ACACA CTTGC GGTTA GCATA CTTTA-3′

Primers for amino acid sequence comprising mutation at site 98:

Upstream primer (SEQ ID NO: 17):
5′-ACCGC AAGTA TGTTC AGCGT TGGTG TA-3′
Downstream primer (SEQ ID NO: 18):
5′-TA CACCA ACGCT GAACA TACTT GCGGT-3′

Vector pET28 comprising SEQ ID NO: 1 was used as a template, and mutant plasmids was amplified by PCR, comprising respectively SEQ ID NO: 19 (SEQ ID NO: 2 N-terminal Cys69 substituted by Ser), SEQ ID NO: 20 (SEQ ID NO: 2 N-terminal Cys72 substituted by Gly), SEQ ID NO: 21 (SEQ ID NO: 2 N-terminal Cys85 substituted by Ser), SEQ ID NO: 22 (SEQ ID NO: 2 N-terminal Cys87 substituted by Arg), SEQ ID NO: 23 (SEQ ID NO: 2 N-terminal His95 substituted by Asn), and SEQ ID NO: 24 (SEQ ID NO: 2 N-terminal 98Cys substituted by Tyr).

2. The entire nucleotide sequences shown in SEQ ID NO: 19-24 were amplified from mutant plasmids with primers as follows:

Upstream primer (SEQ ID NO: 5):
5′-CGCGGATCC ATGGCTGATCATTTGAGTTTATGT-3′,
Downstream primer (SEQ ID NO: 6):
5′-CCGGAGCTC TCAAACTGGTGTTGGGACATTGGATA-3′.

PCR products were purified (see manuals from Qiagen corp.), enzymatic digested by BamH1 and Sac1, recovered from the gel, and ligated into the vector pBI121 (ligation sites: BamH1 and Sac1), to obtain over expression recombinant plasmid comprising SEQ ID NO: 19-24, which were named MT1-6, respectively.

### Example 3

### Characterization of the Heat-Resisting Capacity of E. coli with Over Expressed AtTR1 and Mutants Thereof

1. E. coli Transformation

E. coli was transformed with recombinant plasmid and plated onto LB agar containing Amp. The E. coli pET28 strain containing recombinant plasmid was obtained after sequencing.

2. Medium Preparation

LB liquid medium was prepared with the addition of antibiotics Kan 50 ug/ml and Cam 50 ug/ml, and then stored in 24 tubes, each with 10 ml.

3. Bacterial Suspension Preparation

Single colonies of E. coli comprising over expressed AtTR1 and the 6 mutant genes (MT1-6) and non-transgenic E. coli were activated overnight at 37° C.

4. Dropwise Addition of Sample Bacteria

0.05 ml of activated bacteria liquid was innoculated into tubes containing LB culture medium, 3 tubes for each bacterium, followed by shaking and cultivating (37° C., 225 rpm) for 3 hours.

5. Culture and Observation

After being incubated at 37° C. for 3 hours, the culture was added with isopropyl β-D-1-thiogalactopyranoside (IPTG) in a concentration of 0.1 mM, and the incubation was continued at 42° C. for 10 h. The growth status of E. coli comprising over expressed AtTR1 and the 6 mutant genes (MT1-6) and non-transgenic E. coli were observed with naked eyes, and OD600 values were determined at regular times (TU-1800 ultraviolet spectrophotometer, Puxitong instrument corp., Beijing, China) to record growth curves of different E. coli, see FIG. 1 (wherein AtTR1 meant E. coli transformed with over expressed AtTR1 gene, MT1-6 respectively meant E. coli transformed with over expressed mutant AtTR1 genes, and WT meant non-transgenic E. coli).

Results showed that, although E. coli transformed with over expressed mutant AtTR1 genes exhibited some heat resistance, the slopes of their growth curves were significantly smaller than that of E. coli with over expressed AtTR1, indicating that heat resistance would be lowered after mutations at the 6 sites and such sites played an important role in the heat-resisting function of the AtTR1 protein in organisms.

### Example 4

### Characterization of the Heat-Resisting Capacity of Brassica Transformed with Over Expressed AtTR1 and Mutants Thereof

1. Transformation of Brassica napus by Hypocotyl Infection

1-1. Obtaining Aseptic Seedlings

Plump Brassica napus seeds were selected and vernalized overnight at 4° C. (synchronized germination), then immersed with 70% of ethanol for 30 s and 0.1% of mercuric chloride (HgCl₂) solution for 8-10 min, rinsed with sterile water for 5 times and dried with filter paper, and then plated onto MS agar medium. After cultivated at 24° C. for 2-3 days in a dark chamber, the culture was exposed to illumination for 16 h/d to continue germination. The hypocotyl of aseptic seedlings (in about 7-8 days) was taken with 5-7 cm as transformation receptors.

1-2. Pre-Culture of the Hypocotyl

The Brassica napus hypocotyl was sliced into sections of about 7 mm and well distributed onto pre-culture medium (MS+2 mg/L of 6-BA, 1 mg/L of 2,4-D, 2.5 mg/L of AgNO₃ and 19.62 mg/L of acetosyringone) to be pre-cultivated for 2-3 days (wherein the hypocotyl became coarse).

1-3. Infection and Co-Cultivation of Hypocotyls

Agrobaterium containing recombinant plasmids comprising over expressed SEQ ID NO: 1 and SEQ ID NOs: 19-24, respectively, were innoculated into LB medium containing 20 mg/L of Str, 50 mg/L of Kan and 40 mg/L of Rif and cultivated overnight at 28° C., and bacteria were collected and resuspended with MS medium containing 100 mg/L acetosyringone till OD₆₀₀=0.4-0.6, followed by incubating at 28° C. for 1-2 h.

Pre-cultured healthy Brassica napus hypocotyls were immersed into bacteria liquid of Agrobaterium containing recombinant plasmids comprising over expressed SEQ ID NO: 1 and SEQ ID NOs: 19-24, respectively, for 30 s-1 min, with constant oscillation to fully contact the bacteria liquid with hypocotyls. Spare bacteria liquid was fastly removed with aseptic filter paper. Then brassica napus hypocotyls were laid flat onto co-culture medium (MS+2 mg/L of 6-BA, 1 mg/L of 2,4-D, 2.5 mg/L of AgNO₃ and 19.62 mg/L of acetosyringone) to be co-cultivated for 2 days.

1-4. Screening Culture and Germ Induction

The seven of co-cultivated Brassica napus hypocotyls were respectively innoculated into differential medium (MS+2 mg/L of 6-BA, 1 mg/L of 2,4-D, 2.5 mg/L of AgNO₃ and 19.62 mg/L of acetosyringone) to continue the cultivation. Germ callus was obtained after 4 weeks of cultivation with the medium refreshed by every 2 weeks.

1-5. Radication

The germ was sliced from callus tissue and transferred onto radication medium (1/2 MS, 0.15 mg/L of NAA and 250 mg/L of Cef) after the germ callus had grown up with 4-6 pieces of euphylla on screening culture medium (MS+2 mg/L of 6-BA, 2.5 mg/L of AgNO₃, 500 mg/L of carbenicillin and 10 mg/L of Kan). The culture tank was moved outdoors for 2-3 d after the root system of regenerated seedlings had grown well, followed by opening the tank and hardening seedlings for 2-3 d.

1-6. Pot Culture

Transgenic plants comprising over expressed SEQ ID NO: 1 and SEQ ID NOs: 19-24 were cultivated respectively on radication medium to develop the entire root system, and then transferred to pot culture.

2. PCR Detection of Transgenic Brassica napus

Total DNA was extracted respectively from small amount of leaves of the regenerated plants that had grown well in soil, and PCR detection was performed with the DNA extracted as templates.

Primers for AtTR1 Detection:

Upstream primer (SEQ ID NO: 25):
5′-TGATCATTTGAGTTTATGTACCGA-3′
Downstream primer (SEQ ID NO: 26):
5′-TCAAACTGGTGTTGGGACATTGGAT-3′

Primers for MT1 Detection:

Upstream primer (SEQ ID NO: 27):
5′-AATCTGTTGAGAGTCGTATTTGCCAA-3′
Downstream primer (SEQ ID NO: 28):
5′-TCAAACTGGTGTTGGGACATTGGAT-3′

Primers for MT2 Detection:

Upstream primer (SEQ ID NO: 29):
5′-GTCGTATTGGCCAAGAGGAAGATAG-3′
Downstream primer (SEQ ID NO: 30):
5′-TCAAACTGGTGTTGGGACATTGGAT-3′

Primers for MT3 Detection:

Upstream primer (SEQ ID NO: 31):
5′-CTTGAAGCTCCTAGTGCTTGTAAT-3′
Downstream primer (SEQ ID NO: 32):
5′-TCAAACTGGTGTTGGGACATTGGAT-3′

Primers for MT4 Detection:

Upstream primer (SEQ ID NO: 33):
5′-CTTGTGCTCGTAATGGTAGTTTAAAGT-3′
Downstream primer (SEQ ID NO: 34):
5′-TCAAACTGGTGTTGGGACATTGGAT-3′

Primers for MT5 Detection:

Upstream primer (SEQ ID NO: 35):
5′-AAGTATGCTAACCGCAAGTGTGTTCT-3′
Downstream primer (SEQ ID NO: 36):
5′-TCAAACTGGTGTTGGGACATTGGAT-3′

Primers for MT6 Detection:

Upstream primer (SEQ ID NO: 37):
5′-TATGCTCACCGCAAGTATGTTCAGCGTTT-3′
Downstream primer (SEQ ID NO: 38):
5′-TCAGACTGGTGTTGGGTTGGATAT-3′

Then agarose electrophoresis was performed to detect the presence of target band (with a size of about 860 bp), which was an indication that the target gene had been transformed into Brassica napus. And after such detections, brassica transformed with relevant genes were obtained.

3. Characterization of Heat Resisting Capacity of Transgenic Brassica—Seed Germinations Under Heat Shock

3-1. Seeds Vernalization

7 kinds of transgenic Brassica napus seeds (AtTR1, MT1, MT2, MT3, MT4, MT5, and MT6) and non-transgenic wild type (WT) were selected, 200 for each, and incubated overnight in water at 4° C.

3-2. Heat Shock Treatment

8 kinds of brassica seeds were respectively loaded into labeled tubes, 2 tubes for each kind, 100 seeds for each tube, and divided into groups A and B, wherein group A was incubated in water at 45° C. for 3 hours, and group B at room temperature for 2 hours.

3-3. Results

After 2 hours, seeds were placed in plates covered with filter paper and cultured in a chamber (illumination of 6,000-8,000 lux, 16 h/8 h light-dark cycles), and observed every 24 hours for the statistics of relative germination rates (relative germination rate=germination rate after heat shock/germination rate under room temperature×100%), see Table 5.

TABLE 5
Relative germination rates of brassica seeds after heat shock (%)

| | AtTR1 | MT1 | MT2 | MT3 | MT4 | MT5 | MT6 | WT |
|---|---|---|---|---|---|---|---|---|
| 1 day later | 0 | 0 | 0 | 0 | 0 | 0 | 0 | 0 |
| 2 days later | 32.48 | 20.00 | 14.68 | 13.75 | 10.06 | 10.11 | 16.94 | 12.82 |
| 3 days later | 68.52 | 41.97 | 38.55 | 35.69 | 30.17 | 26.96 | 40.00 | 32.22 |
| 4 days later | 74.13 | 57.43 | 41.26 | 44.20 | 42.88 | 40.25 | 47.86 | 40.85 |
| 5 days later | 84.00 | 62.35 | 47.87 | 50.00 | 51.25 | 47.01 | 49.96 | 46.87 |
| 6 days later | 86.17 | 69.02 | 56.95 | 62.75 | 60.77 | 59.20 | 62.45 | 59.50 |
| 7 days later | 93.55 | 70.38 | 65.82 | 70.13 | 69.00 | 62.85 | 68.29 | 64.00 |

4. Characterization of Transgenic Brassica Heat Resisting Capacity—Growth Status at the Seedling Stage at Heat Shock

7 kinds of transgenic Brassica napus seeds (AtTR1, MT1, MT2, MT3, MT4, MT5, and MT6) and non-transgenic wild type (WT) were respectively placed on moist filter papers for germination. After shell breaking, they were transferred into humus soil and cultured at 22° C. for about 15 days (with the growth of two pieces of euphylla), then transferred into heat stress of 34° C. under conditions of 14 h in sunlight and 10 h in dark. Growth status of each brassica was observed.

Results showed that, after 3 days of heat stress, the growth of Brassica napus was inhibited, wherein the transgenic MT1-6 and wild type all exhibited yellow and withered leaves, wherein MT1, MT2 and MT6 exhibited less yellowing and witheredness, and WT, MT3, MT4 and MT5 were relatively more severe, while AtTR1 grew normally. After 5 days of heat stress, MT1-6 and the wild type were all dead, while AtTR1 survived and grew normally.

### Example 5

### Obtaining of the RcTR1 Gene, a Homologous Gene of AtTR1 from Castor Oil Plant, and Heat Resistance Experiment of E. coli Transformed with RcTR1

1. Cloning and Obtaining of Homologous Gene from Castor Oil Plant

The castor oil plant total RNA was extracted using TRIzol method (Invitrogen Corp., TRIzol agent) and reverse transcripted into cDNA (promega Corp., Reverse transcription kit). Primers were designed according to the RcTR1 nucleotide sequence from castor oil plant shown in SEQ ID NO: 39 (the amino acid sequence encoded by which is shown in SEQ ID NO: 40):

Upstream primer (SEQ ID NO: 41):
5′-GTGGTGTAGCAGGATTTTTAATC-3′
Downstream primer (SEQ ID NO: 42):
5′-GCATCCCCATTCATTTCAT-3′

Nucleotides 37-964 from SEQ ID NO: 39 were amplified from castor oil plant cDNA. The PCR product was purified (see manuals from Qiagen corp.) and sequenced to obtain the desired sequence.

2. Primers with restriction enzyme cutting site were designed according to the forementioned amplified sequence fragments, and the recombinant plasmid was constructed.

Upstream primer (SEQ ID NO: 43):
5′-CCGGAATTCATGAGTGATCAACTAGTTTTG-3′
Downstream primer (SEQ ID NO: 44):
5′-CCCAAGCTTTCATTGAAGTGGCTCTTG-3′

Nucleotides 68-946 from SEQ ID NO: 39 were amplified from the PCR product of step 1 (the amino acid sequence encoded by which is shown in SEQ ID NO: 40). The PCR product was purified (see manuals from Qiagen corp.) and digested with EcoR1 and HindIII. After being recovered from gels, fragments were respectively ligated into vector pET28 to obtain recombinant plasmids. The recombinant plasmids were transformed into E. coli JM109 and plated onto LB agar containing kanamycin, and sequenced to obtain E. coli JM109 strain containing recombinant plasmid.

3. After the E. coli JM109 strain was cultured, plasmid was extracted (see manuals from Qiagen corp.) and transformed into E. coli BL21 Rosset cells and plated onto LB agar containing kanamycin and chloramphenicol. Single colonies were picked and innoculated into LB medium containing 100 mg/L of kanamycin and chloramphenicol, and cultured overnight at 37° C. and 225 rpm. After cultivated, the culture was innoculated by 1: 250 into fresh LB medium (containing 100 mg/L of kanamycin and chloramphenicol) and OD600 value was detected (TU-1800 ultraviolet spectrophotometer, Puxitong instrument corp., Beijing, China). Different cultures were adjusted to the same OD value, and then subjected to shaking culture (37° C., 225 rpm) for 3 h, followed by the addition of 1 mM of IPTG, OD detection, and shaking culture again (42° C., 225 rpm, 10 h), wherein the OD value was detected every hour.

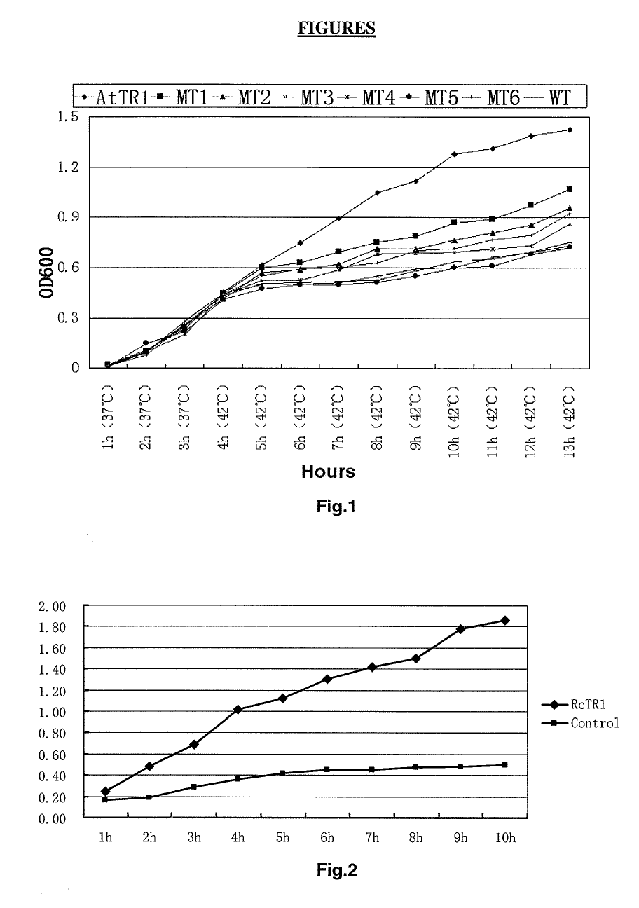

Results showed that, E. coli transformed with castor oil plant homologous gene RcTR1 exhibited higher heat resistance than non-transgenic E. coli. As was shown in FIG. 2, higher OD values stood for higher concentration of bacteria, i.e., better growth status. Varied with time, the growth curve slope of E. coli transformed with castor oil plant homologous gene (RcTR1) was significantly higher than that of non-transgenic E. coli (control), indicating that the growth rate and status of E. coli transformed with castor oil plant homologous gene RcTR1 was significantly better than that of non-transgenic E. coli.

### Example 6

### Obtaining of Genes OsTR1-1 and OsTR1-2, Homologous Genes of AtTR1 from Rice, and Heat Resistance Experiment of E. coli Transformed with OsTR1-1 and OsTR1-2

1. Cloning and Obtaining of Homologous Genes OsTR1-1 and OsTR1-2 from Rice

The rice total RNA was extracted using TRIzol method (Invitrogen Corp., TRIzol agent) and reverse transcripted into cDNA (promega Corp., Reverse transcription kit). Primers were designed respectively according to nucleotide sequences shown in SEQ ID NO: 45 (the amino acid sequence encoded by which is shown in SEQ ID NO: 46) and SEQ ID NO: 47 (the amino acid sequence encoded by which is shown in SEQ ID NO: 48):

Primers for the amplification of OsTR1-1 sequence (SEQ ID NO: 45):

Upstream primer (SEQ ID NO: 49):
5′-AAACTTTTTTGGGTGATTTGC-3′
Downstream primer (SEQ ID NO: 50):
5′-CAAATTCTATTGCCCTTGTTCT-3′

Primers for the amplification of OsTR1-2 sequence (SEQ ID NO: 57):

Upstream primer (SEQ ID NO: 51):
5′-GGAATTTGGGATGGGCGACCA-3′
Downstream primer (SEQ ID NO: 52):
5′-AGAGACGCTACTGAAGTAACTAGCTAT-3′

Nucleotides 71-854 from OsTR1-1 sequence (SEQ ID NO: 45) (the amino acid sequence encoded by which is shown in SEQ ID NO: 46), and nucleotides 281-1150 from OsTR1-2 sequence (SEQ ID NO: 47) (the amino acid sequence encoded by which is shown in SEQ ID NO: 48) were respectively amplified from rice cDNA. The PCR product was purified (see manuals from Qiagen corp.) and sequenced to obtain the desired sequence.

2. Primers with restriction enzyme cutting sites were designed according to the forementioned amplified sequence fragments, and the recombinant plasmid was constructed.

Primers for OsTR1-1 sequence (SEQ ID NO: 45) containing restriction enzyme cutting sites:

Upstream primer (SEQ ID NO: 53):
5′-CGCGGATCCATGGGGGATCATGTTGCGGTGGAT-3′
Downstream primer (SEQ ID NO: 54):
5′-CCGGAGCTCCTATTGCCCTTGTTCTGGATGAGGT-3′

Primers for OsTR1-2 sequence (SEQ ID NO: 47) containing restriction enzyme cutting sites:

Upstream primer (SEQ ID NO: 55):
5′-CGCGGATCCATGGGCGACCATGTGGTGGT-3′
Downstream primer (SEQ ID NO: 56):
5′-CCGGAGCTCGACGCTACTGAAGTAACTAGCTAT-3′

Expression sequences were amplified from the PCR product of step 1 (sites 96-848 in SEQ ID NO: 45, sites 291-1147 in SEQ ID NO: 47). The PCR product was purified (see manuals from Qiagen corp.) and digested with BamH1 and Xhol1. After being recovered from gels, fragments were respectively ligated into vector pET28 to obtain recombinant plasmids. The recombinant plasmids were transformed into E. coli JM109 and plated onto LB agar containing kanamycin, and sequenced to obtain E. coli JM109 strain containing recombinant plasmid.

3. After the E. coli JM109 strain was cultured, plasmid was extracted (see manuals from Qiagen corp.) and transformed into E. coli BL21 Rosset cells and plated onto LB agar containing kanamycin and chloramphenicol. Single colonies were picked and innoculated into LB medium containing 100 mg/L of kanamycin and chloramphenicol, and cultured overnight at 37° C. and 225 rpm. After cultivated, the culture was innoculated by 1: 250 into fresh LB medium (containing 100 mg/L of kanamycin and chloramphenicol) and OD600 value was detected (TU-1800 ultraviolet spectrophotometer, Puxitong instrument corp., Beijing, China). Different cultures were adjusted to the same OD value, and then subjected to shaking culture (37° C., 225 rpm) for 3 h, followed by the addition of 1 mM of IPTG, OD detection, and shaking culture again (42° C., 225 rpm, 10 h), wherein the OD value was detected every hour.

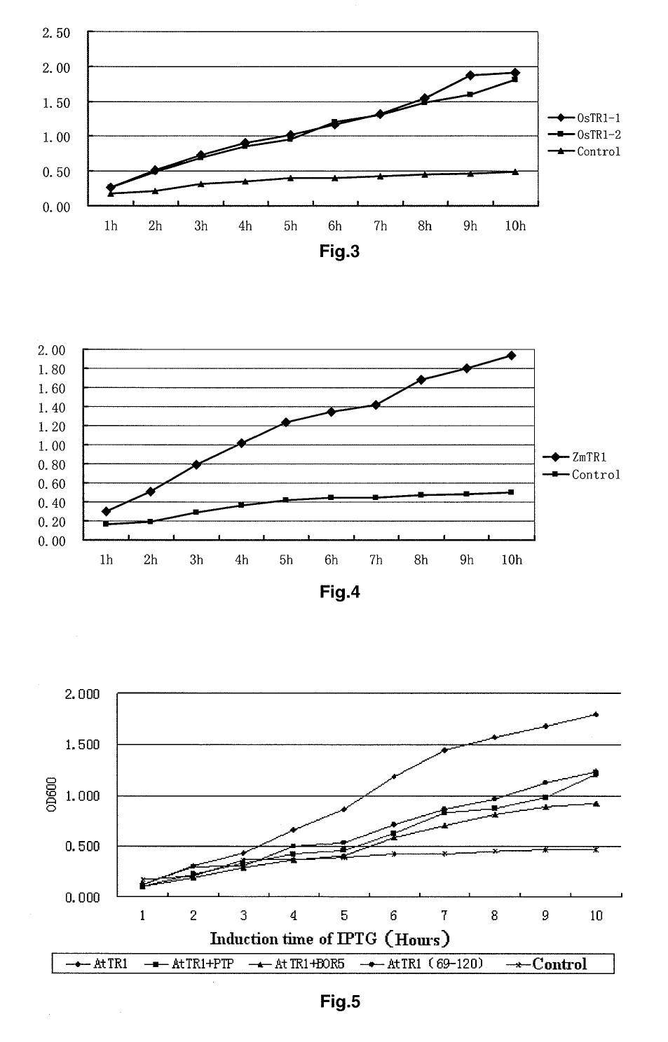

Results showed that, E. coli transformed with the two rice homologous genes OsTR1-1 and OsTR1-2 exhibited higher heat resistance than non-transgenic E. coli. As was shown in FIG. 3, higher OD values stood for higher concentration of bacteria, i.e., better growth status. Varied with time, the growth curve slope of E. coli transformed with rice homologous gene (OsTR1-1 and OsTR1-2) was significantly higher than that of non-transgenic E. coli (control), indicating that the growth rate and status of E. coli transformed with rice homologous gene OsTR1-1 and OsTR1-2 was significantly better than that of non-transgenic E. coli.

### Example 7

### Obtaining of the ZmTR1 Gene, a Homologous Gene of AtTR1 from Corn, and Heat Resistance Experiment of E. coli Transformed with ZmTR1

1. Cloning and Obtaining of Corn Homologous Gene ZmTR1

The corn total RNA was extracted using TRIzol method (Invitrogen Corp., TRIzol agent) and reverse transcripted into cDNA (promega Corp., Reverse transcription kit). Primers were designed according to SEQ ID NO: 57 from corn (the amino acid sequence encoded by which is shown in SEQ ID NO: 58):

Upstream primer (SEQ ID NO: 59):
5′-TTGGGATGGCTGGTGACG-3′
Downstream primer (SEQ ID NO: 60):
5′-GTGAGCAACTACTGGGGATGTG-3′

Nucleotides 153-1051 from SEQ ID NO: 54 were amplified from corn cDNA. The PCR product was purified (see manuals from Qiagen corp.) and sequenced to obtain the desired sequence.

2. Primers with restriction enzyme cutting sites were designed according to the forementioned amplified sequence fragments, and the recombinant plasmid was constructed.

Upstream primer (SEQ ID NO: 61):
5′-CCGGAATTCATGGCTGGTGACGACC-3′
Downstream primer (SEQ ID NO: 62):
5′-CCCAAGCTTCTATTGCTGTTGCTGCGAC-3′

Expression sequences were amplified from the PCR product of step 1 (sites 158-1018 in SEQ ID NO: 54, the amino acid sequence encoded by which is shown in SEQ ID NO: 58). The PCR product was purified (see manuals from Qiagen corp.) and digested with EcoR1 and HindIII. After being recovered from gels, fragments were respectively ligated into vector pET28 to obtain recombinant plasmids. The recombinant plasmids were transformed into E. coli JM109 and plated onto LB agar containing kanamycin, and sequenced to obtain E. coli JM109 strain containing recombinant plasmid.

3. After the E. coli JM109 strain was cultured, plasmid was extracted (see manuals from Qiagen corp.) and transformed into E. coli BL21 Rosset cells and plated onto LB agar containing kanamycin and chloramphenicol. Single colonies were picked and innoculated into LB medium containing 100 mg/L of kanamycin and chloramphenicol, and cultured overnight at 37° C. and 225 rpm. After cultivated, the culture was innoculated by 1: 250 into fresh LB medium (containing 100 mg/L of kanamycin and chloramphenicol) and OD600 value was detected (TU-1800 ultraviolet spectrophotometer, Puxitong instrument corp., Beijing, China). Different cultures were adjusted to the same OD value, and then subjected to shaking culture (37° C., 225 rpm) for 3 h, followed by the addition of 1 mM of IPTG, OD detection, and shaking culture again (42° C., 225 rpm, 10 h), wherein the OD value was detected every hour.

Results showed that, E. coli transformed with corn homologous gene ZmTR1 exhibited higher heat resistance than non-transgenic E. coli. As was shown in FIG. 4, higher OD values stood for higher concentration of bacteria, i.e., better growth status. Varied with time, the growth curve slope of E. coli transformed with corn homologous gene (ZmTR1) was significantly higher than that of non-transgenic E. coli (control), indicating that the growth rate and status of E. coli transformed with corn homologous gene ZmTR1 was significantly better than that of non-transgenic E. coli.

### Example 8

### Studies on the AtTR1 Structure

In this example, PTP and BOR5 transmembrane domains were used as a substitution of AtTR1 intrinsic transmembrane domain (amino acids 180-239 in SEQ ID NO.2), and AtTR1 intrinsic transmembrane domain was deleted; in order to study the heat resistance of AtTR1, AtTR1 with substituted transmembrane domain, and transmembrane-domain-deleted AtTR1 comprising the RINGv structure only (sites 69-120 in SEQ ID NO.2, the amino acid sequence of which was shown in SEQ ID NO.63).

1. Amplification of Base Sequences Encoding Protein Fragments

Since the forementioned two transmembrane domains were both derived from arabidopsis, the arabidopsis cDNA was used as a template, and base sequences encoding two transmembrane domains were amplified by PCR, respectively. Meanwhile, base sequences encoding amino acids 1-179 and 69-120 in SEQ ID NO.2 were also amplified by PCR from the arabidopsis cDNA.

2. Vector Construction the Obtaining of Recombinant Bacteria

The forementioned amplified gene fragments as well as the pET28a vector was respectively digested with EcoRI and Ecl136II, recovered from the gel, and ligated and transformed into E. coli DH5a. Positive clones were picked and plasmids were extracted, transformed into E. coli Rosseta cells and plated onto LB agar containing kanamycin and chloramphenicol. Single colonies were picked and 4 recombinant bacteria were obtained as follows:

Recombinant bacteria comprising the entire AtTR1 protein, named AtTR1;

Recombinant bacteria with AtTR1 intrinsic transmembrane domain substituted by PTP transmembrane domain, named AtTR1+PTP;

Recombinant bacteria with AtTR1 intrinsic transmembrane domain substituted by BOR5 transmembrane domain, named AtTR1+BOR5;

Recombinant bacteria comprising AtTR1 (amino acids 69-120), i.e., comprising the RINGv structure only and without transmembrane domain, named AtTR1 (69-120).

3. Detection of Heat Resistance

Single colonies were picked and innoculated into LB medium containing 50 mg/L of kanamycin and 34 mg/L of chloramphenicol, and cultured overnight at 37° C. and 225 rpm. After cultivated, the culture was innoculated by 1: 100 into fresh LB medium (containing 50 mg/L of kanamycin and 34 mg/L of chloramphenicol) and cultivated at 37° C. and 225 rmp for 2 hours, and OD600 value was detected (TU-1800 ultraviolet spectrophotometer, Puxitong instrument corp., Beijing, China) till about 0.2. IPTG was then added to a final concentration of 1 mM and cultivation was continued at 42° C. and 225 rpm with shaking, wherein the OD value was detected every hour, and the growth curve was made according to OD values. The growth curve was shown in Table 6 and FIG. 5.

TABLE 6
Growth curves (OD600 values)

| | | | | | Blank |
|---|---|---|---|---|---|
| IPTG | | AtTR1 + | AtTR1 + | AtTR1 | vector |
| induction times | AtTR1 | PTP | BOR5 | (69-120) | control |
| 1 hour | 0.115 | 0.102 | 0.100 | 0.114 | 0.167 |
| 2 hours | 0.300 | 0.217 | 0.184 | 0.293 | 0.199 |
| 3 hours | 0.429 | 0.329 | 0.291 | 0.300 | 0.363 |
| 4 hours | 0.659 | 0.420 | 0.366 | 0.495 | 0.366 |
| 5 hours | 0.863 | 0.459 | 0.401 | 0.529 | 0.387 |
| 6 hours | 1.185 | 0.622 | 0.585 | 0.711 | 0.421 |
| 7 hours | 1.441 | 0.824 | 0.700 | 0.862 | 0.426 |
| 8 hours | 1.566 | 0.866 | 0.814 | 0.963 | 0.449 |
| 9 hours | 1.680 | 0.975 | 0.885 | 1.126 | 0.461 |
| 10 hours | 1.796 | 1.208 | 0.923 | 1.233 | 0.460 |

As was shown in the result, groups expressing the entire AtTR1 protein exhibited the best heat resistance, significantly higher than those of AtTR1+PTP, AtTR1+BOR5, AtTR1 (69-120) and blank vector control. Groups expressing substituted transmembrane domains, namely AtTR1+PTP, AtTR1+BOR5 and AtTR1 (69-120), were possessed with some heat resistance, though significantly lower than that of AtTR1 group. The blank vector control exhibited the worst heat resistance and could hardly grow under the high temperature of 42° C.

### Example 9

### Studies on the Structure of OsTR1-1, the Rice Homologous Protein of AtTR1

In this example, PTP and BOR5 transmembrane domains were used as a substitution of OsTR1-1 intrinsic transmembrane domain (amino acids 145-203 of the protein synthesized by SEQ ID NO.45), and OsTR1-1 intrinsic transmembrane domain was deleted; in order to study heat resistance of OsTR1-1, OsTR1-1 with substituted transmembrane domain, and transmembrane-domain-deleted OsTR1-1 comprising the RINGv structure only (amino acids 32-81 of the protein synthesized by SEQ ID NO.45, the amino acid sequence of which was shown in SEQ ID NO.66).

The experiment procedure was the same as Example 8, and 4 recombinant bacteria were obtained, as follows:

Recombinant bacteria comprising the entire OsTR1-1 protein, named OsTR1-1;

Recombinant bacteria with OsTR1-1 intrinsic transmembrane domain substituted by PTP transmembrane domain, named OsTR1-1+PTP;

Recombinant bacteria with OsTR1-1 intrinsic transmembrane domain substituted by BOR5 transmembrane domain, named OsTR1-1+BOR5;

Recombinant bacteria comprising OsTR1-1 (amino acids 32-81), i.e., comprising the RINGv structure only and without transmembrane domain, named OsTR1-1 (32-81).

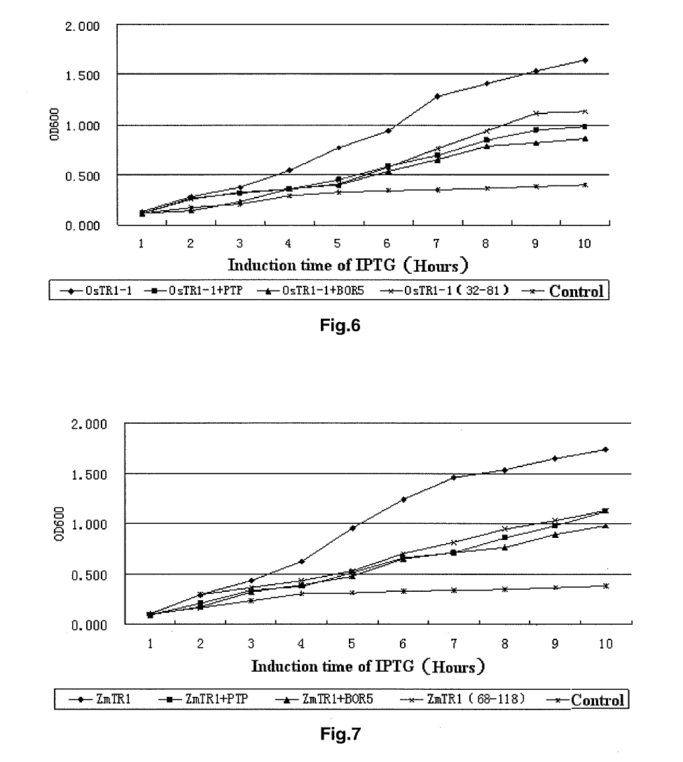

After shaking cultivated with 1 mM of IPTG at 42° C. and 225 rpm for 10 hours, the growth curve was shown in Table 7 and FIG. 6.

TABLE 7
Growth curves (OD600 values)

| | | | | | Blank |
|---|---|---|---|---|---|
| IPTG | | OsTR1- | OsTR1- | OsTR1- | vector |
| inducing times | OsTR1-1 | 1 + PTP | 1 + BOR5 | 1 (32-81) | control |
| 1 hour | 0.127 | 0.110 | 0.109 | 0.110 | 0.106 |
| 2 hours | 0.275 | 0.263 | 0.136 | 0.251 | 0.168 |
| 3 hours | 0.369 | 0.312 | 0.231 | 0.318 | 0.204 |
| 4 hours | 0.538 | 0.356 | 0.351 | 0.357 | 0.289 |
| 5 hours | 0.768 | 0.449 | 0.399 | 0.403 | 0.317 |
| 6 hours | 0.933 | 0.584 | 0.531 | 0.575 | 0.338 |
| 7 hours | 1.280 | 0.694 | 0.649 | 0.759 | 0.345 |
| 8 hours | 1.411 | 0.841 | 0.782 | 0.938 | 0.359 |
| 9 hours | 1.538 | 0.949 | 0.821 | 1.114 | 0.381 |
| 10 hours | 1.645 | 0.981 | 0.860 | 1.129 | 0.394 |

As was shown in the result, groups expressing the entire OsTR1-1 protein exhibited the best heat resistance; significantly higher than those of OsTR1-1+PTP, OsTR1-1+BOR5, OsTR1-1 (32-81) and blank vector control. Groups expressing substituted transmembrane domains, namely OsTR1-1+PTP, OsTR1-1+BOR5 and OsTR1-1 (32-81), were possessed with some heat resistance, though significantly lower than that of OsTR1-1 group. The blank vector control exhibited the worst heat resistance and could hardly grow under the high temperature of 42° C.

### Example 10

### Studies on the Structure of ZmTR1, the Corn Homologous Protein of AtTR1

In this example, PTP and BOR5 transmembrane domains were used as a substitution of ZmTR1 intrinsic transmembrane domain (amino acids 175-234 of the protein synthesized by SEQ ID NO.57), and ZmTR1 intrinsic transmembrane domain was deleted; in order to study heat resistance of ZmTR1, ZmTR1 with substituted transmembrane domain, and transmembrane-domain-deleted ZmTR1 comprising the RINGv structure only (amino acids 68-118 of the protein synthesized by SEQ ID NO. 57, the amino acid sequence of which was shown in SEQ ID NO.65).

The experiment procedure was the same as Example 8, and 4 recombinant bacteria were obtained, as follows:

Recombinant bacteria comprising the entire ZmTR1 protein, named ZmTR1;

Recombinant bacteria with ZmTR1 intrinsic transmembrane domain substituted by PTP transmembrane domain, named ZmTR1+PTP;

Recombinant bacteria with ZmTR1 intrinsic transmembrane domain substituted by BOR5 transmembrane domain, named ZmTR1+BOR5;

Recombinant bacteria comprising ZmTR1 (amino acids 68-118), i.e., comprising the RINGv structure only and without transmembrane domain, named ZmTR1 (68-118).

After shaking cultivated with 1 mM of IPTG at 42° C. and 225 rpm for 10 hours, the growth curve was shown in Table 8 and FIG. 7.

TABLE 8
Growth curves (OD600 values)

| | | | | | Blank |
|---|---|---|---|---|---|
| IPTG | | ZmTR1 + | ZmTR1 + | ZmTR1 | vector |
| inducing times | ZmTR1 | PTP | BOR5 | (68-118) | control |
| 1 hour | 0.108 | 0.100 | 0.100 | 0.106 | 0.102 |
| 2 hours | 0.300 | 0.208 | 0.177 | 0.294 | 0.161 |
| 3 hours | 0.436 | 0.341 | 0.319 | 0.368 | 0.239 |
| 4 hours | 0.622 | 0.372 | 0.389 | 0.439 | 0.305 |
| 5 hours | 0.955 | 0.513 | 0.481 | 0.527 | 0.317 |
| 6 hours | 1.243 | 0.665 | 0.653 | 0.706 | 0.328 |
| 7 hours | 1.458 | 0.711 | 0.714 | 0.814 | 0.339 |
| 8 hours | 1.542 | 0.865 | 0.765 | 0.949 | 0.352 |
| 9 hours | 1.656 | 0.981 | 0.894 | 1.034 | 0.362 |
| 10 hours | 1.741 | 1.118 | 0.986 | 1.131 | 0.380 |

As was shown in the result, groups expressing the entire ZmTR1 protein exhibited the best heat resistance; significantly higher than those of ZmTR1+PTP, ZmTR1+BOR5, ZmTR1 (68-118) and blank vector control. Groups expressing substituted transmembrane domains, namely ZmTR1+PTP, ZmTR1+BOR5 and ZmTR1 (68-118), were possessed with some heat resistance, though significantly lower than that of ZmTR1 group. The blank vector control exhibited the worst heat resistance and could hardly grow under the high temperature of 42° C.

### Example 11

### Characterization of Heat Resistance of Arabidopsis Over Expressed with AtTR1, OsTR1-2, ZmTR1 and RcTR1 Genes and Critical Fragments Thereof

Arabidopsis was transformed with AtTR1 gene (SEQ ID NO: 1), OsTR1-2 gene (SEQ ID NO: 47), ZmTR1 gene (SEQ ID NO: 57) and RcTR1 gene (SEQ ID NO: 39); and critical fragments thereof encoding peptides comprising SEQ ID NO: 63, SEQ ID NO: 67, SEQ ID NO: 65 and SEQ ID NO: 64, in order to study the heat resistance thereof.

Vectors were constructed according to the method in Example 2, and arabidopsis was transformed via in floral dip method, with steps as follows:

1. Agrobaterium containing over expression recombinant plasmids comprising SEQ ID NO: 1, SEQ ID NO: 47, SEQ ID NO: 57, SEQ ID NO: 39, SEQ ID NO: 63, SEQ ID NO: 67, SEQ ID NO: 65 and SEQ ID NO: 64 were innoculated into LB medium containing 20 mg/L of Str, 50 mg/L of Kan and 40 mg/L of Rif, and cultivated overnight at 28° C. Cells were collected and resuspended with MS medium containing 0.01% of surfactant silwet-77 till OD₆₀₀=0.4-0.6, followed by incubating at 28° C. for 1-2 h and keeping the bacteria liquid.

2. The inflorescence of Arabidopsis thaliana cultured for 60 days was cut off and immersed with the bacteria liquid of Agrobaterium containing recombinant plasmids for 2 min. After cultivating in dark for 48 hours, the result Arabidopsis thaliana seedlings were transferred in a normal illumination environment, with resulted legumes as the transgenic TO generation of the seed.

3. The resulted seeds were planted and after 50 days of growth, positive plants were identified via PCR, as follows:

Transgenic arabidopsis comprising AtTR1 (SEQ ID NO: 1), named AtTR1,

Transgenic arabidopsis comprising AtTR1 critical peptides (SEQ ID NO: 63) encoding sequence, named R-AtTR1,

Transgenic arabidopsis comprising OsTR1-2 (SEQ ID NO: 47), named At-OsTR1-2,

Transgenic arabidopsis comprising OsTR1-2 critical peptides (SEQ ID NO: 67) encoding sequence, named At-R-OsTR1-2,

Transgenic arabidopsis comprising ZmTR1 (SEQ ID NO: 57), named At-ZmTR1,

Transgenic arabidopsis comprising ZmTR1 critical peptides (SEQ ID NO: 65) encoding sequence, named At-R-ZmTR1,

Transgenic arabidopsis comprising RcTR1 (SEQ ID NO: 39), named At-RcTR1,

Transgenic arabidopsis comprising RcTR1 critical peptides (SEQ ID NO: 64) encoding sequence, named At-R-RcTR1,

After the maturation of transgenic plants, seeds were collected for reservation.

4. Germination of seeds under heat stress

MS culture medium preparations (see Table 8, with pH adjusted to 5.8 with KOH) were separately loaded into plates after being sterilized with high-pressure steam. After solidified, the culture medium was innoculated with 2 ml of seeds suspended in sterile water, the spare of which was removed after seeding well. The plate cover was opened to dry the medium surface at sterile environment for 1 h. The plate was sealed.

The experiment was divided into two groups. The first group served as the heat shock group, wherein the plate was cultivated in a culture chamber (50° C., illumination intensity 6,000-8,000 1×, 16 h/8 h light-dark cycles, relative humidity 70%) to be treated with heat shock for 2 hours, then subjected to room temperature (22° C.; intensity of illumination, 6,000-8,000 1×; light-dark cycles, 16 h/8 h; relative humidity 70%). Germination and other phenotypes were observed every day for the statistics of germination number. And the second group was a non-heat shock control, wherein the plate was cultivated in a culture chamber (22° C., illumination intensity 6,000-8,000 1×, 16 h/8 h light-dark cycles, relative humidity 70%). Germination and other phenotypes were observed every day for the statistics of germination number, see Table 9.

TABLE 8
Components of MS culture medium

| | | Conc. |
|---|---|---|
| Components | | (mg/L) |
| Major | Potassium nitrate (KNO₃) | 1900 |
| elements | Ammonium nitrate (NH₄NO₃) | 1650 |
| | Potassium dihydrogen phosphate (KH₂PO₄) | 170 |
| | Magnesium sulfate (MgSO₄•7H₂O) | 370 |
| | Calcium chloride (CaCl₂•2H₂O) | 440 |
| Minor | Potassium iodide (KI) | 0.83 |
| elements | Boric acid (H₃BO₃) | 6.2 |
| | Manganese sulfate (MnSO₄•4H₂O) | 22.3 |
| | Zinc sulfate (ZnSO₄•7H₂O) | 8.6 |
| | Sodium molybdate (Na₂MoO₄•2H₂O) | 0.25 |
| | Copper sulfate (CuSO₄•5H₂O) | 0.025 |
| | Cobalt chloride (CoCl₂•6H₂O) | 0.025 |
| Ferric salts | Disodium ethylenediamine tetraacetic acid | 37.3 |
| | Ferrous sulfate (FeSO₄•7H₂O) | 27.8 |
| Organic | Inositol | 100 |
| components | Glycine | 2 |
| | Thiamine hydrochloride (VB1) | 0.1 |
| | Pyridoxine hydrochloride (VB6) | 0.5 |
| | Nicotinic acid (VB5 or VPP) | 0.5 |
| | Sucrose | 30 g/L |
| | Agar | 7 g/L |

TABLE 9
Germination rates of arabidopsis seeds after heat shock

| | Day 5 at 22° C. after | |
|---|---|---|
| | heat shock in 50° C. | Day 5 without heat shock |
| AtTR1 | 74% | 100% |
| R-AtTR1 | 62% | 100% |
| At-OsTR1-2 | 56% | 96% |
| At-R-OsTR1-2 | 40% | 98% |
| At-ZmTR1 | 60% | 100% |
| At-R-ZmTR1 | 42% | 98% |
| At-RcTR1 | 48% | 100% |
| At-R-RcTR1 | 30% | 100% |
| WT | 6% | 98% |

Results showed that, the germination rates of arabidopsis seeds were affected after heat shock treatment, and either the transgenic type or non-transgenic control exhibited lower germination rate after heat shock than those under room temperature. Comparisons indicated that, transgenic arabidopsis AtTR1, At-OsTR1-2, At-ZmTR1 and At-RcTR1, which were transformed with the entire AtTR1, OsTR1-2, ZmTR1 and RcTR1 genes, were affected the least by heat shocks with germination rates of 74%-48%. Transgenic arabidopsis R-AtTR1, At-R-OsTR1-2, At-R-ZmTR1 and At-R-RcTR1, which were transformed with the critical sequences of AtTR1, OsTR1-2, ZmTR1 and RcTR1, were affected less by heat shocks (better than non-transgenic) with germination rates of 62%-30%. While the germination rate of the non-transgenic was merely 6%.

Arabidopsis after heat shock treatment was continued to be cultivated under conditions of 22° C., illumination intensity 6,000-8,000 1×, 16 h/8 h light-dark cycles, and relative humidity of 70%. Results showed that, as time passed, the non-transgenic control grew no green base leaves, with yellow or white seedlings and finally died; while transgenic ones could exhibit green base leaves with good growth status, and could blossom and breed.

Results indicated that, genes of AtTR1, OsTR1-2, ZmTR1 and RcTR1, either entire sequences (SEQ ID NO: 1, SEQ ID NO: 47, SEQ ID NO: 57 and SEQ ID NO: 39) or critical sequences (SEQ ID NO: 63, SEQ ID NO: 67, SEQ ID NO: 65 and SEQ ID NO: 64), could all enhance heat resistance of plants, leading to good germination and growth.

### Example 12

### E3 Ubiquitin Ligase Activity of Proteins AtTR1, OsTR1-1 and ZmTR1

20 ul of reaction systems were prepared, comprising: 1 ug E1 protein, 2 ug E2 protein, 4 ug AtTR1 protein (or OsTR1-1 protein, or ZmTR1 protein), 0.1 mmol/L of ubiquitin (Ub), 2 mmol/L of ATP, 5 mmol/L of MgCl₂, and 50 mmol/L of Tris at pH7.5. Reaction was performed at 30° C. for 90 min, and then 6×SDS-PAGE loading buffer was added to terminate the reaction. After boiling for 5 min, SDS-PAGE electrophoresis was performed.

After electrophoresis, the product was electro-transferred to polyvinylidene difluoride (PVDF) membrane, and Western Blot detection was performed with anti-Ub monoclonal antibody (purchased from Santa Cruz) and alkaline phosphatase-conjugated secondary rat antibody (AP, purchased from Promega).

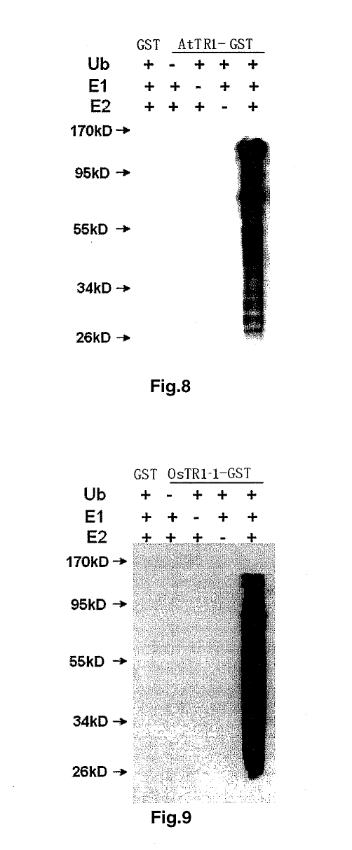

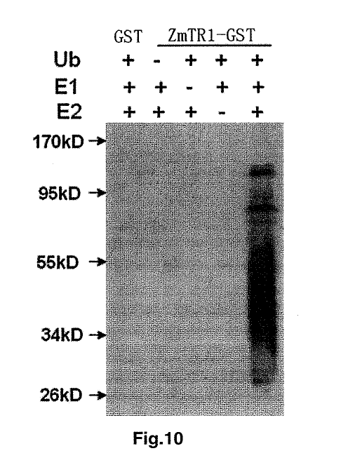

As shown in FIGS. 8, 9 and 10, with the co-functioning of proper E1, E2, ATP and Ub in protein reactions in vitro, Ub could be conjugated with proteins AtTR1, OsTR1-1 or ZmTR1 and form poly-chains. As was shown, multiple bands occurred in the lane with the addition of E1, E2, ATP and Ub, while no band occurred in the control, indicating that proteins AtTR1, OsTR1-1 and ZmTR1 were possessed with ubiquitin E3 ligase activity and could catalyze the formation of monomer or poly-ubiquitins.

As was shown in the forementioned examples, protein AtTR1 of the invention and homologous proteins thereof in plants such as corn, rice and castor oil plant showed capability of enhancing heat resistance of plants or microorganisms. Research showed that, such kind of proteins all comprised zinc finger of RINGv type, and belonged to E3 ligase. The invention had also found the critical region of the protein—RINGv zinc finger, wherein E3 ligase proteins comprising such a structure would enhance heat resistance of plants or microorganisms, while proteins being deleted with the structure would totally lose the function. In addition, the transmembrane domain was studied for the protein, and results showed that proteins comprising the intrinsic transmembrane domain could better enhance heat resistance of plants or microorganisms, and proteins with transmembrane domain substituted by others could still exhibit some heat resistance, better than that of non-transgenic type. To sum up, RINGv zinc finger was the critical peptide structure of AtTR1 proteins that led to the heat resistance, said structure comprising N′-CRICQE X₇₋₄₅ PCAC X₆ AHR X₁ CVQ X₁₃₋₂₇-C′ or further comprising N′-CRICQEED X₃₋₂₀ NL X₃₋₂₀ PCAC X₂ SLK X₁ AHR X₁ CVQRWC X₁₀₋₂₄-C′ (wherein X represents any amino acid and the subscript represents the number of amino acid). The invention thus provided new and promising alternative genes encoding heat-resisting proteins or peptides, and proteins or peptides encoded by the same, as well as uses of these genes, proteins and peptides for the enhancement of heat resistance of plants and microorganisms.

1-43. (canceled)

44. A gene encoding a protein able to enhance heat resistance of plants or microorganisms, wherein said gene comprises a nucleotide sequence encoding a peptide as follows:
N′-CRICQE X₇₋₄₅ PCAC X₆ AHR X₁ CVQ X₁₃₋₂₇-C′ (SEQ ID NO: 68), wherein X represents any amino acid and the subscript represents the number of amino acid, further wherein said protein is an ubiquitin ligase.

45. The gene encoding a protein able to enhance heat resistance of plants or microorganisms according to claim 44, wherein said peptides are: N′-CRICQEED X₃₋₂₀ NL X₃₋₂₀ PCAC X₂ SLK X₁ AHR X₁ CVQRWC X₁₀₋₂₄-C′(SEQ ID NO: 69), wherein X represents any amino acid and the subscript represents the number of amino acid.

46. The gene encoding a protein able to enhance heat resistance of plants or microorganisms according to claim 44, wherein said protein is a transmembrane protein.

47. The gene encoding a protein able to enhance heat resistance of plants or microorganisms according to claim 44, wherein said protein further comprises transmembrane domains.

48. The gene encoding a protein able to enhance heat resistance of plants or microorganisms according to claim 47, wherein said protein comprises 1-6 transmembrane domains.

49. The gene encoding a protein able to enhance heat resistance of plants or microorganisms according to claim 47, wherein said protein comprises 2-3 transmembrane domains.

50. The gene a encoding protein able to enhance heat resistance of plants or microorganisms according to claim 47, wherein said transmembrane domains comprise at least one structure as shown in N′-A X₂₋₆ CRS X₂₋₈ LIL X₂₋₄ LL X₁₋₄ LR X₁₋₁₀-C′ (SEQ ID NO: 70) or N′-L X₂₋₄ R X₁₋₅ GFLL X₁₋₇ YIMAW X₁₋₁₅-C′ (SEQ ID NO:71), wherein X represents any amino acid and the subscript represents the number of amino acids.

51. The gene encoding a protein able to enhance heat resistance of plants or microorganisms according to claim 47, wherein said protein further comprises a signal peptide.

52. The gene encoding a protein able to enhance heat resistance of plants or microorganisms according to claim 51, wherein said signal peptide is a membrane localization signal peptide.

53. The gene encoding a protein able to enhance heat resistance of plants or microorganisms according to claim 44, wherein said gene is derived from a plant genome.

54. The gene encoding a protein able to enhance heat resistance of plants or microorganisms according to claim 47, wherein said plant is selected from the group consisting of arabidopsis, rice, corn, and castor oil plant.

55. The gene encoding a protein able to enhance heat resistance of plants or microorganisms according to claim 44, wherein (i): said gene comprises at least one nucleotide sequence selected from the group consisting of SEQ ID NO: 39, SEQ ID NO: 1, SEQ ID NO: 45, SEQ ID NO: 47, SEQ ID NO: 57, and combinations thereof;
Or (ii): said gene comprises nucleotide sequences derived from substitution, deletion or addition of at least one nucleotide in any one of said nucleotide sequences in (i), while encoding proteins with the same or similar function.

56. The gene encoding a protein able to enhance heat resistance of plants or microorganisms according to claim 53, wherein (i): said gene comprises at least one nucleotide sequence selected from the group consisting of sites 158-1018 in SEQ ID NO: 57, sites 96-848 in SEQ ID NO: 45, sites 291-1127 in SEQ ID NO: 47, sites 68-946 in SEQ ID NO: 39, and combinations thereof;
Or (ii): said gene comprises nucleotide sequences derived from substitution, deletion or addition of at least one nucleotide in any one of said nucleotide sequences in (i), while encoding proteins with the same or similar function.

57. A protein able to enhance heat resistance of plants or microorganisms, wherein said protein comprise peptides as follows:
N′-CRICQE X₇₋₄₅ PCAC X₆ AHR X₁ CVQ X₁₃₋₂₇-C′ (SEQ ID NO: 68), wherein X represents any amino acid and the subscript represents the number of amino acid; further wherein said protein is an ubiquitin ligase.

58. The protein able to enhance heat resistance of plants or microorganisms according to claim 57, wherein said peptides comprise structures of: N′-CRICQEED X₃₋₂₀ NL X₃₋₂₀ PCAC X₂ SLK X₁ AHR X₁ CVQRWC X₁₀₋₂₄-C′ (SEQ ID NO: 69), wherein X represents any amino acid.

59. The protein able to enhance heat resistance of plants or microorganisms according to claim 57, wherein said protein further comprises transmembrane domains.

60. the protein able to enhance heat resistance of plants or microorganisms according to claim 59, wherein said protein further comprises 1-6 transmembrane domains.

61. The protein able to enhance heat resistance of plants or microorganisms according to claim 60, wherein said protein comprises 2-3 transmembrane domains.

62. The protein able to enhance heat resistance of plants or microorganisms according to claim 59, wherein said transmembrane domains comprise structures shown in at least one of N′-A X₂₋₆ CRS X₂₋₈ LIL X₂₋₄ LL X₁₋₄ LR X₁₋₁₀-C′ (SEQ ID NO: 70) or N′-L X₂₋₄ R X₁₋₅ GFLL X₁₋₇ YIMAW X₁₋₁₅-C′ (SEQ ID NO: 71), wherein X represents any amino acid and the subscript represents the number of amino acid.

63. The protein able to enhance heat resistance of plants or microorganisms according to claim 57, wherein said protein further comprises a signal peptide.

64. The protein able to enhance heat resistance of plants or microorganisms according to claim 63, wherein said signal peptide is a membrane localization signal peptide.

65. The protein able to enhance heat resistance of plants or microorganisms according to claim 57, wherein said protein is derived from plants.

66. The protein able to enhance heat resistance of plants or microorganisms according to claim 57, wherein (i) said protein comprises amino acid sequences encoded by nucleotide sequences selected from the group consisting of SEQ ID NO: 1, sites 158-1018 in SEQ ID NO: 57, sites 96-848 in SEQ ID NO: 45, sites 291-1127 in SEQ ID NO: 47, sites 68-946 in SEQ ID NO: 39, and combinations thereof;
Or (ii): said protein comprises amino acid sequences derived from substitution, deletion or addition of at least one amino acid in any one of amino acid sequences in (i), while possessing with the same or similar function.

67. A gene encoding said protein able to enhance heat resistance of plants or microorganisms according to claim 57.

68. A peptide which comprises domains as shown in N′-CRICQE X₇₋₄₅ PCAC X₆ AHR X₁ CVQ X₁₃₋₂₇ (SEQ ID NO: 68), wherein X represents any amino acid and the subscript represents the number of amino acid; said peptide enables proteins to enhance heat resistance of plants or microorganisms.

69. The peptide according to claim 68, wherein said peptide comprises domains as shown in N′-CRICQEED X₃₋₂₀ NL X₃₋₂₀ PCAC X₂ SLK X₁ AHR X₁ CVQRWC X₁₀₋₂₄-C′ (SEQ ID NO: 69), wherein X represents any amino acid and the subscript represents the number of amino acid.

70. The peptide according to claim 68, wherein it is derived from plants.

71. The peptide according to claim 68, wherein said plant is selected from the group consisting of arabidopsis, rice, corn, and castor oil plant.

72. The peptide according to claim 68, wherein it can form a zinc finger.

73. The peptide according to claim 68, wherein it comprises polypeptide sequences as follows:
(i): at least one amino acid sequences selected from the group consisting of shown in SEQ ID NO: 63, SEQ ID NO: 64, SEQ ID NO: 65, SEQ ID NO: 66, SEQ ID NO: 67 and combinations thereof;
Or, (ii) amino acid sequences derived from substitution, deletion or addition of at least one amino acid in any one of polypeptides in (i), while possessing with the same or similar function.

74. Nucleotide sequences encoding any one of said peptides in claim 68.

75. A method of modifying genes encoding non-heat-resisting proteins into genes encoding heat-resisting proteins, comprising the steps of inserting nucleotide sequences encoding a peptide of claim 68 into genes encoding non-heat-resisting proteins, or substituting one section of nucleotide sequences therein; wherein such an insertion or substitution occurs between codons, wherein the modified gene still has the ability to encode an entire protein.

76. The method according to claim 75, wherein the nucleotide sequence substituted in said gene encoding non-heat-resisting protein is the nucleotide sequence encoding zinc finger domain.

77. The method according to claim 75, wherein said non-heat-resisting protein is an ubiquitin ligase.

78. The method for enhancing plant heat resistance or preparing plants with high heat resistance, comprising the steps of:
a. Operably linking said gene of claim 44 with expression regulation sequence of a vector to form a recombinant vector expressing said gene;
b. Transforming the recombinant vector of step (a) into plant cells;
c. Selecting and obtaining transformed cells, followed by regenerating the transformed cells to form transgenic plants or offspring thereof, wherein said offspring include plant seeds or plant tissues.

79. Vectors comprising the gene of claim 44 that encodes a protein able to enhance heat resistance of plants or microorganisms.

80. The vector according to claim 79, wherein said vector is an expression vector.

81. The vector according to claim 80, wherein said expression vector is eukaryotic.

82. Host cells comprising the gene of claim 44 that encodes protein able to enhance heat resistance of plants or microorganisms.

83. The host cells according to claim 80, wherein said host cells are plant cells or microorganisms.

84. A method for enhancing the tolerance of plants or microbes to stresses comprising transforming plants with peptides having amino acid sequences of N′-CRICQE X₇₋₄₅ PCAC X₆ AHR X₁ CVQ X₁₃₋₂₇-C′ (SEQ ID NO: 68), protein comprising the peptides, or a gene comprising nucleotide sequences encoding the peptides or protein,; wherein X represents any amino acid and the subscript represents the number of amino acid.

85. The method according to claim 84, wherein said peptides are: N′-CRICQEED X₃₋₂₀ NL X₃₋₂₀ PCAC X₂ SLK X₁ AHR X₁ CVQRWC X₁₀₋₂₄-C′ (SEQ ID NO: 69), wherein X represents any amino acid and the subscript represents the number of amino acid.

86. The method according to claim 84, wherein said peptides have a function of ubiquitin ligase.

87. The method according to claim 84, wherein said protein is a transmembrane protein.

88. The method according to claim 87, wherein said protein further comprises transmembrane domains.

89. The method according to claim 88, wherein said protein comprises 1-6 transmembrane domains.

90. The method according to claim 89, wherein said protein comprises 2-3 transmembrane domains.

91. The method according to claim 88, wherein said transmembrane domains comprise at least one structure shown in N′-A X₂₋₆ CRS X₂₋₈ LIL X₂₋₄ LL X₁₋₄ LR X₁₋₁₀-C′ (SEQ ID NO: 70) or N′-L X₂₋₄ R X₁₋₅ GFLL X₁₋₇ YIMAW X₁₋₁₅-C′ (SEQ ID NO:71), wherein X represents any amino acid and the subscript represents the number of amino acid.

92. The method according to claim 87, wherein said protein further comprises a signal peptide.

93. The method according to claim 92, wherein said signal peptide is a membrane localization signal peptide.

94. The method according to claim 84, wherein said gene is derived from a plant genome.

95. The method according to claim 94, wherein said plant is selected from the group consisting of arabidopsis, rice, corn, and castor oil plant.

96. The method according to claim 84, wherein (i): said gene comprises nucleotide sequences selected from the group consisting of sites 158-1018 in SEQ ID NO: 57, sites 96-848 in SEQ ID NO: 45, sites 291-1127 in SEQ ID NO: 47, sites 68-946 in SEQ ID NO: 39, and combinations thereof;
Or (ii): said gene comprises nucleotide sequences derived from substitution, deletion or addition of at least one nucleotide in any one of said nucleotide sequences in (i), while encoding proteins with the same or similar function.

97. The method according to claim 84, wherein (i): said gene comprises nucleotide sequences selected from the group consisting of SEQ ID NO: 39, SEQ ID NO: 1, SEQ ID NO: 45, SEQ ID NO: 47, and SEQ ID NO: 57;
Or (ii): said gene comprises nucleotide sequences derived from substitution, deletion or addition of at least one nucleotide in any one of said nucleotide sequences in (i), while encoding proteins with the same or similar function.

98. The method according to claim 84, wherein (i) said peptides or protein comprises at least one amino acid sequence encoded by nucleotide sequences selected from the group consisting of SEQ ID NO: 63, SEQ ID NO: 64, SEQ ID NO: 65, SEQ ID NO: 66, SEQ ID NO: 67, and combinations thereof;
Or (ii): said peptides or protein comprises amino acid sequences derived from substitution, deletion or addition of at least one amino acid in any one of amino acid sequences in (i), while possessing with the same or similar function.

99. The method according to claim 84, wherein (i) said peptides or protein comprises amino acid sequences encoded by nucleotide sequences selected from the group consisting of SEQ ID NO: 1, sites 158-1018 in SEQ ID NO: 57, sites 96-848 in SEQ ID NO: 45, sites 291-1127 in SEQ ID NO: 47, and sites 68-946 in SEQ ID NO: 39;
Or (ii): said peptides or protein comprises nucleotide sequences derived from substitution, deletion or addition of at least one amino acid in any one of amino acid sequences in (i), while possessing with the same or similar function.
